(19) United States
(12) Patent Application Publication
(10) Pub. No.: US 2011/0295375 A1
(43) Pub. Date: Dec. 1, 2011

(54) Allogenic Articular Cavity Prosthesis and Method for Implanting the Same

(76) Inventors: Andreas Appenzeller, Langendorf (CH); Christian Gerber, Zumikon (CH); Philippe Gedet, Langendorf (CH); Daniel Fluri, Langendorf (CH); Mazda Farshad, Gockkausen (CH)

(21) Appl. No.: 13/080,294

(22) Filed: Apr. 5, 2011

Related U.S. Application Data
(60) Provisional application No. 61/348,950, filed on May 27, 2010.

(51) Int. Cl. A61F 2/30; A61B 17/56

(52) U.S. Cl. 623/18.11; 606/87

(57) ABSTRACT

An articular cavity prosthesis comprises a block extending along a central axis and including a top section and a base section, the top section having an articular bearing surface extending transverse to the central axis and a supporting surface opposite the articular bearing surface configured to contact a surface of a bone on which the block is to be mounted, the base section having a fixing protrusion protruding outward from the supporting surface along the central axis and having a volume V, the block being formed of human tissue.

### PRIORITY CLAIM

The present application claims priority to U.S. Provisional Application Ser. No. 61/348,950 filed on May 27, 2010 and entitled “Allogenic Articular Cavity Prosthesis and Method for Implanting the Same,” the entire disclosure of which is incorporated herein by reference.

### FIELD OF THE INVENTION

The present invention generally relates to articular cavity prostheses and methods for implanting the same. More particularly, the present invention relates to allogenic articular cavity prostheses and a method for implanting the same. Exemplary embodiments of the present invention describe a method for producing an allogenic articular cavity prosthesis and a cutting instrument used for producing the allogenic articular cavity prosthesis.

### BACKGROUND

Currently, damaged shoulder joints are generally treated by insertion of partial prostheses. The damaged cartilage is removed via a special instrument to subsequently produce a base for implantation of the prosthesis using suitable drill sleeves. One problem associated with the known treatment using hemi- or total shoulder arthroplasty is that glenoid erosion or development of progressive glenoid arthritis can result in pain or failure of the hemi-arthroplasty. Further, loosening of the prosthetic glenoid component with compromised scapular neck bone stock is the main failure mode of total shoulder arthroplasty, particularly in young patients. Furthermore, known techniques for biological resurfacing of the glenoid have so far failed to adequately restore the geometry, the biology and the longevity of the articulating glenoid. Thus, there remains a need for an improved prosthesis allowing adequate restoration of the geometry, biology and longevity of the articulating glenoid and to prevent loosening of the prosthetic glenoid component.

### SUMMARY OF THE INVENTION

It is therefore an object of the present invention to provide a prosthesis for an articular cavity replacement, particularly for the shoulder joint, allowing adequate restoration of the geometry, biology and longevity of the hemi-arthroplasty while preventing loosening and/or failure of the hemi-arthroplasty.

[0005]The present invention relates to an articular cavity prosthesis, particularly for replacement of the articular cavity of a shoulder joint, comprising a block having a central axis and including a top section and a base section. The top section includes an articular bearing surface transverse to the central axis and a supporting surface opposite thereto for contacting a surface of a bone. The base section has a fixing protrusion extending coaxially with the central axis and has a volume V. The block is preferably formed of material comprising human tissue.

The prosthesis according to the invention (including its fixation means) is formed of material comprising human tissue, particularly of allogenic cartilage material that grows together with the patient's bone. The risk of glenoid erosion and/or the development of glenoid arthritis is therefore reduced along with a corresponding reduction in pain and a lower risk of failure of the prosthesis. The prosthesis comprises an articular bearing shell allowing adequate restoration of the geometry, biology and longevity of the articulating glenoid. Also, due to growing together of the material of the prosthesis and the bone, rigid fixation of the prosthesis in the bone can be achieved. Thus, the risk of loosening of the prosthetic glenoid component is reduced.

In an exemplary embodiment, the fixing protrusion has a noncircular cross- sectional area orthogonal to the central axis. Due to the noncircular cross-section of the fixing protrusion, the articular cavity prosthesis is prevented from rotating relative to the bone. Therefore, the prosthesis may be provided with a single fixing protrusion. A configuration of the prosthesis with only one fixing protrusion simplifies manufacturing and a design with only one voluminous fixing protrusion is particularly advantageous in case of a prosthesis formed of a human tissue material. The cross-sectional area of the fixing protrusion orthogonal to the central axis may have the form of, for example, an ellipse, oval, wedge, a segment of a circle with a flattened section or a polygon with rounded corners. The fixing protrusion may be, for example, cylindrical, conical or prismatic.

In a further exemplary embodiment, the block consists of natural cartilage, preferably of allogenic cartilage.

In a further exemplary embodiment, the fixing protrusion is formed unitarily with the articular cavity prosthesis. The one-piece configuration increases the rigidity of the prosthesis.

In a still further embodiment, the concave articular bearing surface is continuous and has no holes. The continuous articular bearing surface is more anatomical and reduces surface pressure and wear.

In another exemplary embodiment, the volume V of the fixing protrusion is at least about 1 cm³, preferably at least about 1.2 cm³. Typically the volume V of the fixing portion is about 1.3 cm³.

In yet another exemplary embodiment, the articular cavity prosthesis has an overall volume V_{O} and a ratio of the volume V of the fixing protrusion to an overall volume V_{O} is at least about 0.15, preferably at least about 0.17.

In again another exemplary embodiment, the articular cavity prosthesis has an overall volume V_{O} and the ratio of the volume V of the fixing protrusion to the overall volume V_{O} is at most about 0.9, preferably at most about 0.875. A typical value for the overall volume V_{O} is about 7.3 cm³ and a typical value of the ratio V/V_{O} is about 0.175.

In a further exemplary embodiment, a height H of the fixing protrusion is at least about 3 mm, preferably at least about 4 mm. A typical value for the height H is about 6.5 mm.

In a further exemplary embodiment, the supporting surface extends from a periphery of the top section towards a periphery of the fixing portion. Preferably, the supporting surface has a constant angle of elevation a in a range between 0° and 10°.

In still a further exemplary embodiment, the prosthesis has only one fixing protrusion.

In a still further exemplary embodiment, wherein a top section has a thickness δ between the supporting surface and the articular bearing surface is measured orthogonal to the supporting surface, the thickness δ is a minimum of about 4 mm, preferably a minimum of about 4.5 mm.

The present invention also relates to a method for implanting the articular cavity prosthesis according to the invention. The method comprises the steps of positioning a Kirschner-wire in an articular cavity of a patient and removing the damaged cartilage at the articular cavity until a planar base plane is produced on the bone along with selecting a suitable articular cavity prosthesis having a concave articular bearing surface and determining the position of the articular cavity prosthesis on the base plane on the bone in combination with producing a recess penetrating into the bone from the base plane and implanting the articular cavity prosthesis into the bone of a patient, wherein the recess is produced in such a way that it matches the fixing protrusion of the articular cavity prosthesis.

Due to the form of the recess matching the fixing protrusion, a wringing fit, an interference fit or a press fit may be provided between the fixing protrusion and the recess so that no further fixation elements for fixation of the prosthesis are necessary. The articular cavity prosthesis can be completely produced and delivered by the manufacturer. The recess can be intra-operatively produced to match the fixing protrusion by using a lancing tool provided with a cutting edge.

In an exemplary embodiment, the recess is produced by positioning a hollow lancing tool on the base plane where the lancing tool has a cutting edge extending along a non-circular closed curve or polygon and a hollow space, the periphery of which is limited by the cutting edge. The periphery of the hollow space is used as a guidance.

In another exemplary embodiment, the recess is produced by excavating and/or scratching out bone material using a chisel and/or a bone skid.

In another exemplary embodiment, the recess is produced by embossing the porous bone using a stamping die allowing a reduction in loss of bone material, a more compact bone structure and faster healing times.

In a further exemplary embodiment, the recess is produced by milling and excavating the corners using a chisel.

In a further exemplary embodiment, the recess has smaller dimensions than the fixing protrusion, preferably by about 0.1 to about 0.4 mm, to form a press fit.

In again a further exemplary embodiment, the selected articular cavity prosthesis does not include a prepared fixing protrusion and the production of the fixing protrusion is performed after producing the recess in the bone. The fixing protrusion with a cross-sectional area matching the cross-sectional area of the recess can be produced intra-operatively by using a die that is provided with cutting edges.

In yet a further exemplary embodiment, the method further comprises the step of positioning a Kirschner-wire in a position on the Saller-line before removing the defect cartilage. The exact position of the point on the Saller-line where the Kirschner-wire is fixed to the bone is surgeon defined and can be the mid-point of the Saller-line.

In another exemplary embodiment, the removing of the damaged cartilage at the articular cavity is performed by using an oscillating oval or elliptical cutting instrument. The oval cutting instrument is guided by means of the Kirschner-wire and oscillates in an angular range of 20°. Since access to the articular cavity is limited there is not enough space to rotate a tool or instrument. By means of the oval cutting instrument the treatment of the articular cavity can be performed in a single working step. The use of a circular cutting instrument would require a user to repeatedly position the cutting tool at the articular cavity.

In another exemplary embodiment, the implantation of the articular cavity prosthesis is performed using a vibratory plunger. With the insertion of the articular prosthesis using a vibratory plunger, an interlocking fit is achieved between the osseous peripheral walls of the fixing protrusion at the articular cavity prosthesis and the recess.

In again another exemplary embodiment, the placement of the Kirschner-wire is performed using a wire positioning instrument which may be aligned with respect to the Saller-line.

In a further exemplary embodiment, the position of the articular cavity prosthesis on the base plane is determined by inserting the fixing protrusion into a hollow die and by producing a marking groove into the base plane using the hollow die. During the production of the recess, the lancing tool is positioned on the base plane by using the marking groove and the recess can be excavated by chiselling, milling or embossing through the hollow of the lancing tool. The recess can have a typical depth of about 6.5 mm.

In still a further exemplary embodiment the position of the articular cavity prosthesis on the base plane is determined by inserting a hollow die into the recess and by producing a marking groove into a lower surface of the prepared articular cavity prosthesis using the hollow die.

In again a further exemplary embodiment, the implanted articular cavity prosthesis comprises human tissue material only.

In accordance with another aspect of the present invention, a method for producing an articular cavity prosthesis is provided which comprises the steps of providing a block consisting of human tissue material and producing the articular bearing surface in the top section along with producing the supporting surface at the transition between the top section and the base section by using an oscillating saw and producing the fixing protrusion on the base section.

In an exemplary embodiment, the supporting surface is produced by using a gage which is provided with a rest for supporting the articular bearing surface and a plurality of fasteners which permit the block to be fixed in such a manner that the fixing protrusion is aligned with a central axis of the gage and wherein the gage comprises guide slots for guiding an oscillating saw blade under a pre-defined angle with respect to a plane orthogonal to the central axis of the gage.

In another exemplary embodiment, each guide slot is arranged at an angle of about 0°, 5° and 10° with respect to a plane orthogonal to the central axis of the gage.

In a further exemplary embodiment, the fixing protrusion in the base section is produced by using a hollow die having a first cutting element with a first cutting edge extending along a noncircular closed curve or polygon coinciding with the periphery of the cross-sectional area of the fixing protrusion and oppositely arranged a second cutting element with a second cutting edge extending along a similar closed curve or polygon as the first cutting edge and coinciding with the periphery of the cross-sectional area of the recess in a bone.

In accordance with again another aspect, a cutting instrument to be used in the method for implanting an articular cavity prosthesis is provided. The cutting instrument cuts a planar base plane on a bone and comprises a driving shaft and a cutting head with an oval or elliptical front face.

In an exemplary embodiment, the oval or elliptical front face includes a plurality of cutting teeth.

In a further exemplary embodiment, the oval or elliptical front face has a long axis, a short axis and a center and the driving shaft has a shaft axis cutting the front face in a point located on the long axis and spaced apart from the center by an eccentricity e. The cutting instrument has a minimum size with a dimension A parallel to the long axis of about 40 mm, a length B parallel to the short axis of about 30 mm and an eccentricity e of about 3 mm. In another embodiment the cutting instrument has a maximum size with a dimension A parallel to the long axis of about 50 mm and a length B parallel to the short axis of about 30 mm.

### BRIEF DESCRIPTION OF THE DRAWINGS

Several embodiments of the invention will be described in the following by way of example and with reference to the accompanying drawings in which:

FIG. 1 illustrates a lateral view of an embodiment of the articular cavity prosthesis according to the invention;

FIG. 2 illustrates a front view of the embodiment of the articular cavity prosthesis of FIG. 1;

FIGS. 3 to 9 and 21 illustrate the steps performed in an embodiment of the method for implanting an allogenic articular cavity prosthesis according to the invention;

FIG. 10 illustrates a sectional view of a human shoulder joint with the embodiment of the articular cavity prosthesis of FIG. 1 implanted;

FIG. 11 illustrates a perspective view of a gage for producing an articular cavity prosthesis according to the invention;

FIG. 12 illustrates a front view of the gage of FIG. 11;

FIG. 13 illustrates a perspective view of a die for producing an articular cavity prosthesis according to the invention;

FIG. 14 illustrates a cross-section of the die of FIG. 13;

FIG. 15 illustrates a front view of a cutting instrument used for producing an articular cavity prosthesis according to the invention;

FIG. 16 illustrates a perspective view of the cutting instrument of FIG. 15;

FIG. 17 illustrates a perspective view of a lancing tool used in an embodiment of the method according to the invention;

FIG. 18 illustrates a top view of an embodiment of the recess in the bone according to an embodiment of the method according to the invention;

FIG. 19 illustrates a top view of another embodiment of the recess in the bone according to an embodiment of the method according to the invention; and

FIG. 20 illustrates a top view of again another embodiment of the recess in the bone according to an embodiment of the method according to the invention.

### DETAILED DESCRIPTION

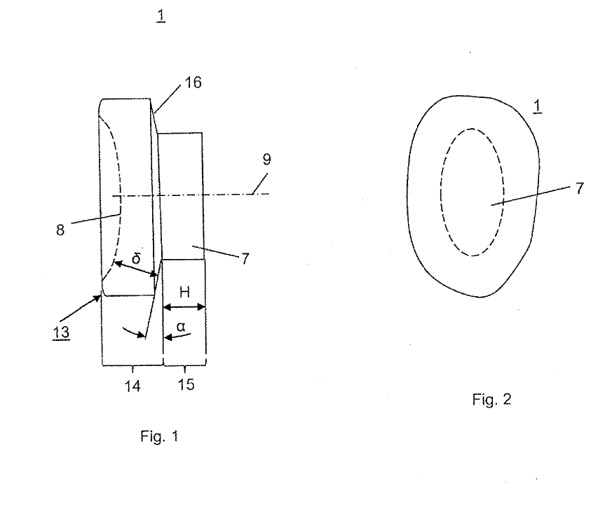

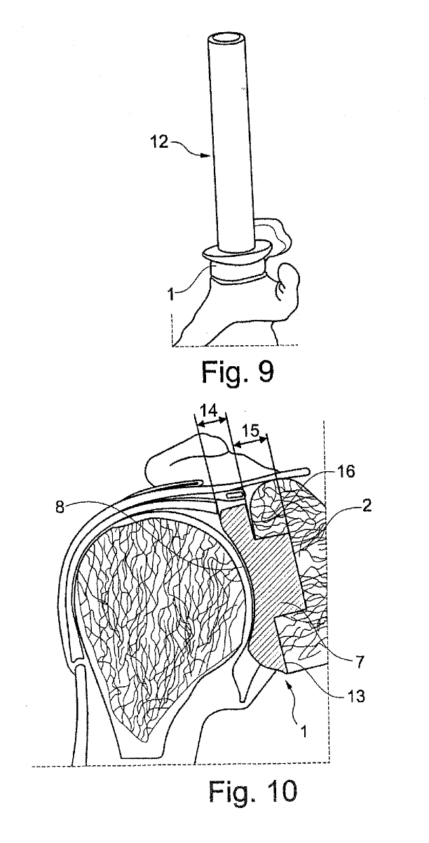

As shown in FIGS. 1-21, a system according to an exemplary embodiment of the present invention comprises an articular cavity prosthesis 1, a cutting tool 10 for producing a base plane 3 in an articular cavity 4 of a bone and a lancing tool 31 for producing a recess 2 in the base plane 3 to receive a portion of the articular cavity prosthesis 1, as described in greater detail below. The system may further comprise a die 17 for marking a desired position of the articular cavity prosthesis 1 on the base plane 3.

FIGS. 1 and 2 illustrate an embodiment of the articular cavity prosthesis 1 including a block 13 formed of allogenic cartilage material, wherein the block 13 extends along a central axis 9 and includes articular bearing surface 8 transverse to the central axis 9 at the opposite side of a fixing protrusion 7 for fixing the prosthesis 1 to a bone. In a preferred embodiment, the block 13 consists entirely of allogenic cartilage material. The allogenic articular cavity prosthesis 1 has a top section 14 with the articular bearing surface 8 on one side and a supporting surface 16 for contacting a surface of a bone on the opposite side. Further, the articular cavity prosthesis 1 has a base section 15 including the fixing protrusion 7. The fixing protrusion 7 extends along the central axis 9 and has a volume V and a height H measured from the supporting surface 16. Further, the fixing protrusion 7 may be substantially cylindrical with a cross-sectional area orthogonal to the central axis 9 in the form of an ellipse. It will be understood by those of skill in the art, however, that the fixing protrusion 7 may be any of a variety of shapes so long as the shape prevents the articular cavity prosthesis 1 from rotating relative to the articular cavity 4 when implanted into the bone. The supporting surface 16 extends from the periphery of the top section 14 towards the periphery of the fixing portion 7 with a constant angle of elevation a of about 5°. The top section 14 has a thickness δ between the supporting surface 16 and the articular bearing surface 8 and measured orthogonal to the supporting surface 16, wherein the thickness δ has a value of 4 mm to 4.5 mm.

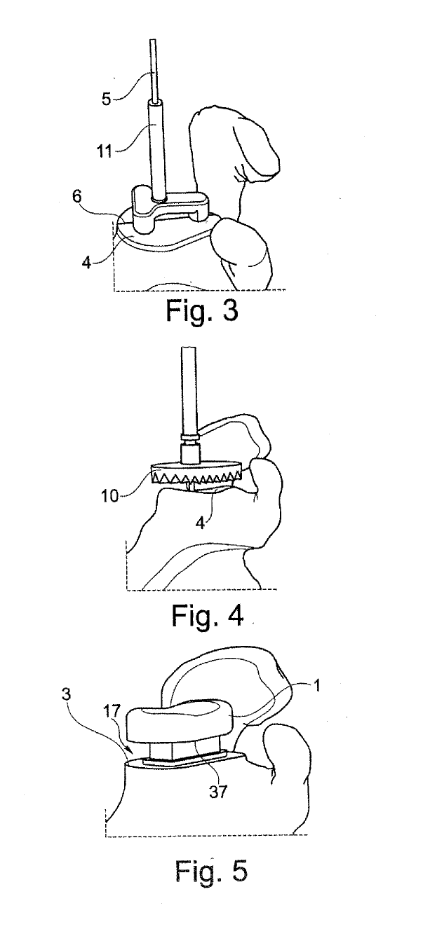

FIGS. 3 to 10 and 21 illustrate an embodiment of the method for implanting an allogenic articular cavity prosthesis 1 comprising the step of positioning a Kirschner-wire 5 in the articular cavity 4, i.e. the articular cavity of a shoulder joint through a surgeon defined point on the Saller-line 6 (FIG. 3). The Saller-line 6 is a vertical line partitioning the articular cavity into an anterior and a posterior half. This step is performed with the use of a particular wire positioning instrument 11 that can be exactly aligned with the Saller line 6 in order to correctly position the Kirschner-wire 5. The defect cartilage at the articular cavity 4 is removed until a planar base plane 3 is produced on the bone by using a cutting, milling or rasping instrument (FIG. 4). As a preferred instrument an oscillating oval or elliptical cutting instrument 10 which has cutting teeth arranged at its front face is used. The oval cutting instrument 10 is guided by the previously implanted Kirschner-wire 5 and oscillates in an angular range of 20°. It will be understood by those of skill in the art, however, that the cutting instrument 10 may include cutting teeth arranged in a variety of shapes, so long as the cutting teeth are arranged to correspond to a shape of the fixing protrusion 7. The mentioned base plane 3 is the basis for the articular cavity prosthesis 1.

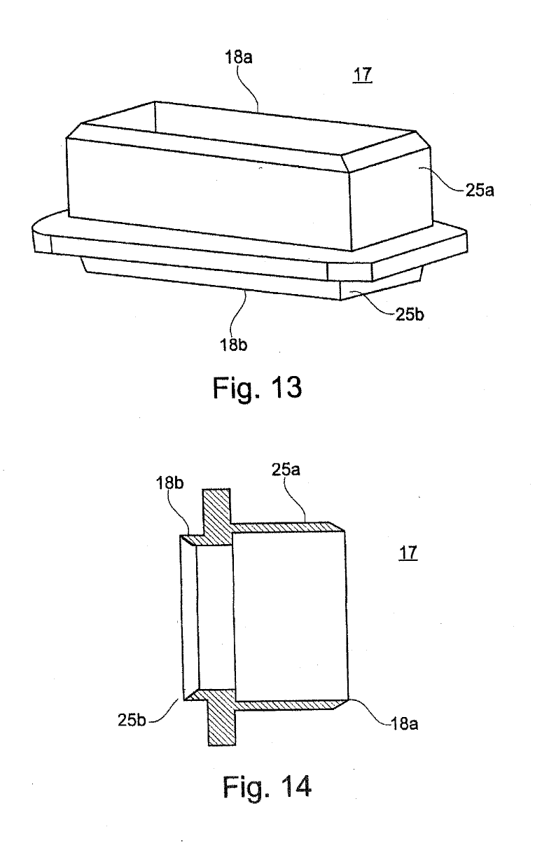

A suitable allogenic articular cavity prosthesis 1 may be selected, the allogenic articular cavity prosthesis 1 having a concave articular bearing surface 8 and oppositely thereto a fixing protrusion 7. The allogenic articular cavity prosthesis 1 can be produced by a manufacturer from a block 13 of allogenic cartilage material (FIG. 1), e.g. on a milling machine. The fixing protrusion 7 is configured with an exactly known shape so that it will fit into a complementarily shaped recess 2 penetrating into the bone from the base plane 3. A position of the articular cavity prosthesis 1 on the base plane 3 on the bone (FIG. 5) is then determined. To exactly position the articular cavity prosthesis 1 a hollow die 17 (FIGS. 13 and 14) is mounted on the fixing protrusion 7 of the articular cavity prosthesis 1 in such a manner that the fixing protrusion 7 in inserted into the first cutting element 25a and the second cutting edge 18b of the second cutting element 25b is positioned on the base plane 3. A marking groove is then produced on the base plane 3 by pressing the second cutting edge 18b of the second cutting element 25b into the bone.

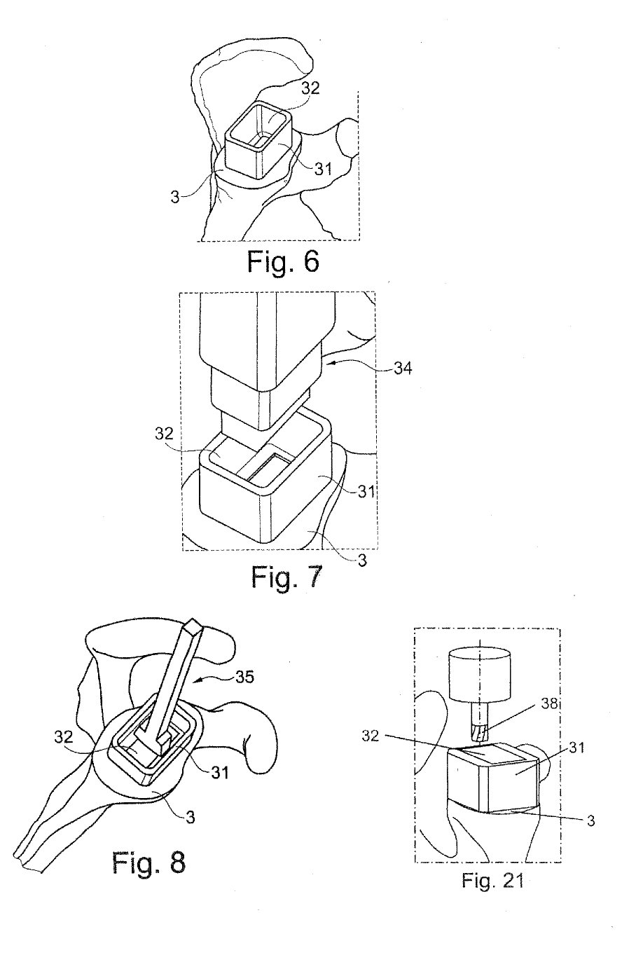

The recess 2 is produced in the bone, wherein the recess 2 has a cross-sectional area that matches the cross-sectional area of the fixing protrusion 7 of the articular cavity prosthesis 1 (FIGS. 6-8). The recess 2 can be prepared e.g. by embossing, chiselling and/or drilling by using a hollow lancing tool 31 and specially adapted instruments, e.g. chisels, cutters or punching instruments to remove the required volume of bone. The recess 2 can have a depth of e.g. 6.5 mm. To produce the recess 2 a hollow lancing tool 31 is positioned on the base plane 3. The lancing tool 31 comprising a cutting edge 33 extending along a non-circular closed curve or polygon and a hollow space 32 the periphery of which is limited by the cutting edge 33. Thus, the cutting edge 33 of the lancing tool 31 can engage the marking groove produced as described above. Further the periphery of the hollow space 32 can be used as a guidance for excavating the recess 2 by embossing, chiselling or milling. Particularly, the recess 2 can be produced by excavating and/or scratching out the bone material using a chisel and/or a bone skid 35 (FIG. 8), by embossing the porous bone using a stamping die 34 (FIG. 7) or by milling with a milling cutter 38 (FIG. 21) and excavating the corners using a chisel;

Once the recess 2 has been formed, the articular cavity prosthesis 1 is implanted into the scapula of a patient. During implantation of the articular cavity prosthesis 1 the fixing protrusion 7 arranged at the articular cavity prosthesis 1 is pressed into the recess 2 by using a vibratory plunger 12 to fix the articular cavity prosthesis 1 to the bone. Due to the use of the vibratory plunger 12 an interlocking fit of the osseous peripheral walls of the fixing protrusion 7 at the articular cavity prosthesis 1 and the recess 2 results.

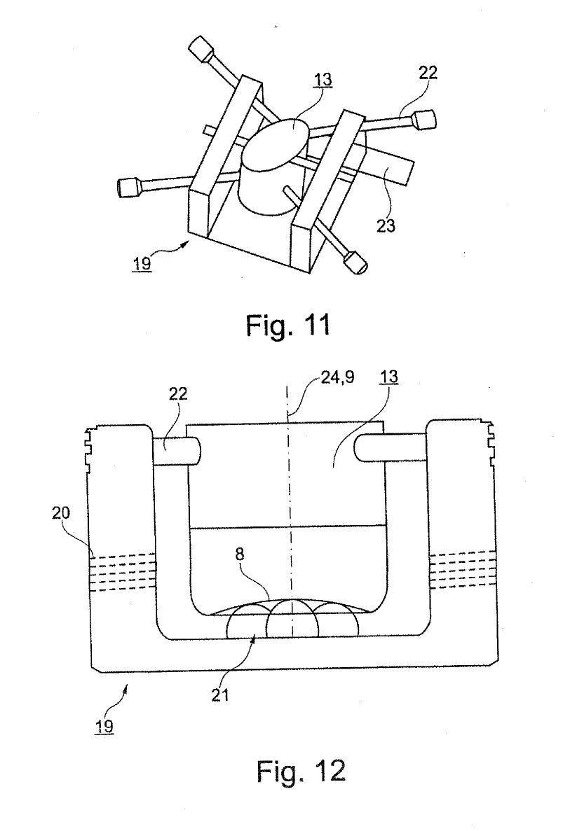

Another embodiment of the method for implanting an allogenic articular prosthesis 1 differs from the embodiment of FIGS. 3 to 10 and 21 only in that a prepared articular cavity prosthesis 1 is provided by the manufacturer without the fixing protrusion 7. The production of the fixing protrusion 7 is performed after producing the recess 2 according to the above step. Further, the position of the articular cavity prosthesis 1 on the base plane 3 is determined by inserting the hollow die 17 into the recess 2 and producing a marking groove into a lower surface 37 of the prepared articular cavity prosthesis 1 using the hollow die 17 (FIG. 5). The fixing protrusion 7 has a cross- sectional area matching the cross-sectional area of the recess 2. The fixing protrusion 7 in the base section 15 is produced by using the hollow die 17 and a saw or wire. Further, the supporting surface 16 is produced by using a gage 19 (FIGS. 11 and 12) which is provided with a rest 21 for supporting the articular bearing surface 8 and a plurality of fasteners 22 which permit the block 13 to be fixed in a manner such that the fixing protrusion 7 is aligned with the central axis 24 of the gage 19. The gage 19 comprises guide slots 20 for guiding an oscillating saw blade 23 under a surgeon selected angle of 0°, 5° or 10° with respect to a plane orthogonal to the central axis 24 of the gage 19.

FIGS. 11 to 14 illustrate particular instruments, e.g., a gage 19 (FIGS. 11 and 12) and a die 17 (FIGS. 13 and 14) used in an embodiment of the method for producing an articular cavity prosthesis 1. The articular cavity prosthesis 1 is produced by providing a block 13 consisting of allogenic cartilage material and producing the articular bearing surface 8 in the top section 14. The supporting surface 16 is produced on the block 13 at the transition between the top section 14 and the base section 15 by using an oscillating saw and the gage 19. The gage 19 is provided with a rest 21 for supporting the articular bearing surface 8 and a plurality of fasteners 22 allowing to fix the block 13 in such a manner that the fixing protrusion 7 is aligned with a central axis 24 of the gage 19. Further, the gage 19 comprises guide slots 20 wherein each a guide slot 20 is arranged at an angle of 0°, 5° and 10° with respect to a plane orthogonal to the central axis 24 of the gage 19. The guide slots 20 are suitable for guiding an oscillating saw blade 23 under a selected angle with respect to a plane orthogonal to the central axis 24 of the gage 19.

The fixing protrusion 7 may be formed on the base section 15. The fixing protrusion 7 in the base section 15 is produced by using a hollow die 17 having a first cutting element 25a with a first cutting edge 18a extending along a rectangular curve with rounded corners coinciding with the periphery of the cross-sectional area of the fixing protrusion 7 and oppositely arranged a second cutting element 25b with a second cutting edge 18b extending along a similar closed curve as the first cutting edge 18a and coinciding with the periphery of the cross-sectional area of the recess 2 in a bone. To produce the fixing protrusion 7 in the base section 15 the first cutting edge 18a of the first cutting element 25a of the hollow die 17 is pressed into a lower surface 37 of the prepared articular cavity prosthesis 1 to produce a marking groove as a guidance for a saw or wire.

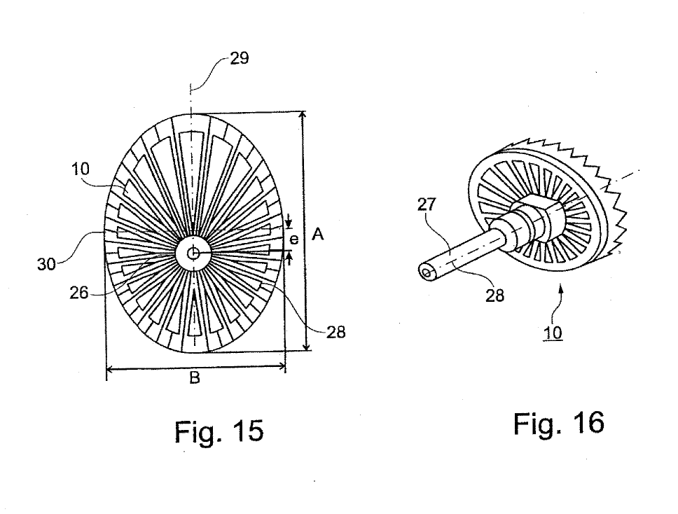

FIGS. 15 and 16 illustrate an oscillating oval or elliptical cutting instrument 10 for producing a plane base plane 3 on a bone. The oval or elliptical cutting instrument 10 comprises a driving shaft 27 and a cutting head with an oval or elliptical front face that includes a plurality of cutting teeth. Further, the oval or elliptical front face has a long axis 29, a short axis 30 and a center 26. The driving shaft 27 has a shaft axis 28 cutting the front face in a point located on the long axis 29 and spaced apart from the center 26 by an eccentricity e. The cutting teeth extend on the front face from the point where the shaft axis 28 cuts the front face to the periphery of the cutting head. Although the cutting instrument 10 is described as oval or elliptical, it will be understood by those of skill in the art that the cutting instrument 10 may include cutting teeth arranged in any shape so long as the arrangement corresponds to a shape of the fixing protrusion 7 of the articular cavity prosthesis 1.

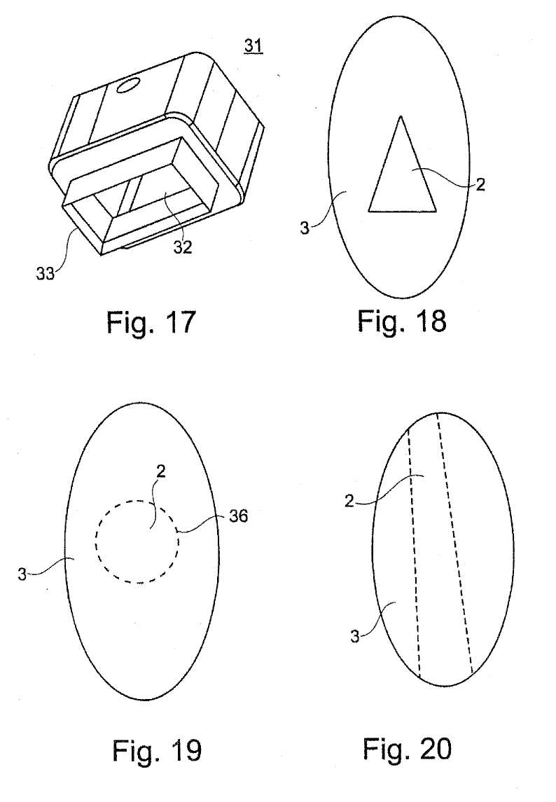

FIG. 17 illustrates an embodiment of a hollow lancing tool 31 comprising a cutting edge 33 extending along a non-circular closed curve or polygon and a hollow space 32 the periphery of which is limited by the cutting edge 33. The cutting edge 33 extends along a closed curve coinciding with the periphery of the cross-sectional area of the recess 2 to be produced in the base plane 3 on the bone, e.g. in the form of an ellipse (FIG. 2), oval, wedge (FIG. 20), a segment of circle with a flattened section 36 (FIG. 19) or a polygon, particularly a triangle with rounded corners (FIG. 18).

Although the invention and its advantages have been described in detail, it should be understood that various changes, substitutions, and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As those of ordinary skill in the art will readily appreciate from the disclosure of the present invention, processes, machines, manufacture, composition of matter, means, methods, or steps, presently existing or later to be developed that perform substantially the same function or achieve substantially the same result as the corresponding embodiments described herein may be utilized according to the present invention.

It will be appreciated by those skilled in the art that various modifications and alterations of the invention can be made without departing from the broad scope of the appended claims. Some of these have been discussed above and others will be apparent to those skilled in the art.

1. An articular cavity prosthesis, comprising a block extending along a central axis and including a top section and a base section, the top section having an articular bearing surface extending transverse to the central axis and a supporting surface opposite the articular bearing surface configured to contact a surface of a bone on which the block is to be mounted, the base section having a fixing protrusion protruding outward from the supporting surface along the central axis and having a volume V, the block being formed of human tissue.

2. The prosthesis according to claim 1, wherein the fixing protrusion has a noncircular cross-sectional area orthogonal to the central axis.

3. The prosthesis according to claim 1, wherein the block is formed of natural cartilage

4. The prosthesis according to claim 1, wherein the block is formed of allogenic cartilage.

5. The prosthesis according to claim 1, wherein the base section is integrally formed with the top section.

6. The prosthesis according to claim 1, wherein the concave articular bearing surface is continuous and has no holes extending therethrough.

7. The prosthesis according to claim 1, wherein V is at least 1 cm³.

8. The prosthesis according to claim 1, wherein the articular cavity prosthesis has an overall volume V_{O} and wherein a ratio V to the overall volume V_{O} is at least 0.15.

9. The prosthesis according to claim 1, wherein the articular cavity prosthesis has an overall volume V_{O} and wherein a ratio of V to the overall volume V_{O} no more than 0.9.

10. The prosthesis according to claim 1, wherein the supporting surface extends from a periphery of the top section towards a periphery of the fixing portion.

11. The prosthesis according to claim 1, wherein the prosthesis has only one fixing protrusion.

12. A method for implanting an articular cavity prosthesis, comprising the steps of:
removing a portion of damaged cartilage from an articular cavity until a planar base plane is produced on a target portion of bone on which a prosthesis is to be mounted;
selecting a suitable articular cavity prosthesis comprising a block extending along a central axis and including a top section and a base section, the top section having a concave articular bearing surface transverse to the central axis and, opposite the bearing surface, a supporting surface configured to contact the target portion of bone, wherein the base section has a fixing protrusion projecting away from the supporting surface along the central axis and having a volume V, the block being formed of human tissue material;
determining a position of the articular cavity prosthesis on the base plane;
producing a recess penetrating into the target portion of bone from the base plane, the recess corresponding in shape and size to the fixing protrusion; and
implanting the articular cavity prosthesis by inserting the fixing protrusion into the recess produced in the target portion of bone.

13. The method according to claim 13, wherein the recess is produced by:
positioning a hollow lancing tool on the base plane, the lancing tool comprising a non-circular, closed cutting edge and a hollow space a periphery of which is limited by the cutting edge; and
using the periphery of the hollow space as a guidance for forming the recess.

14. The method according to claim 12, wherein the recess is produced by embossing porous bone using a stamping die.

15. The method according to claim 12, wherein the recess is smaller than the fixing protrusion by approximately 0.1-0.4 mm such that the recess and the fixing protrusion form a press fit.

16. The method according to claim 12, further comprising the step of positioning of a Kirschner-wire to the bone, preferably in a position on the Saller-line before removing the defect cartilage.

17. The method according to claim 12, wherein the removing the damaged cartilage from the articular cavity is performed by using an oscillating cutting instrument including cutting teeth corresponding to a shape of the fixing protrusion.

18. The method according to claim 12, wherein the implantation of the articular cavity prosthesis is performed using a vibratory plunger.

19. A method for producing an articular cavity prosthesis, comprising the steps of:
i) providing a block formed of human tissue material, the block extending along a central axis and including a top section and a base section;
ii) producing an articular bearing surface in the top section of the block;
iii) producing a supporting surface at a transition between the top section and the base section using an oscillating saw; and
iv) producing a fixing protrusion on the base section, the fixing protrusion extending from the supporting surface along the central axis.

20. The method according to claim 19, wherein the fixing protrusion is produced using a hollow die having a first cutting element with a first cutting edge coinciding with a periphery of a cross-sectional area of the fixing protrusion and an oppositely arranged second cutting element with a second cutting edge coinciding with a periphery of a cross-sectional area of a recess in a target portion of bone into which the protrusion is to be inserted.

21. A system for implanting an articular cavity prosthesis, comprising:
a cutting instrument for producing a base plane, the cutting instrument including a drive shaft and a cutting head;
a hollow lancing tool for producing a recess in the base plane, the hollow lancing tool including a channel extending therethrough for receiving a tool configured to remove a volume of the bone, the channel sized and shaped to correspond to a desired size and shape of the recess; and
an articular cavity prosthesis comprising a block extending along a central axis and including a top section and a base section, the top section having an articular bearing surface transverse to the central axis and, opposite thereto, a supporting surface configured to contact a surface of a target portion of bone adjacent to the recess, the base section having a fixing protrusion extending away from the supporting surface along the central axis and having a volume V, wherein the block consists of human tissue material.

22. The system of claim 21, further comprising a hollow die for marking a desired position of the articular cavity prosthesis in the base plane, the hollow die including a cutting edge extending about a periphery thereof for forming a marking groove when pressed against the base plane.
